(19) United States
(12) Patent Application Publication
(10) Pub. No.: US 2004/0106909 A1
(43) Pub. Date: Jun. 3, 2004

(54) Device for incontinence and method of use

(76) Inventor: Robert L. Browning, Benton Harbor, MI (US)

Correspondence Address: MCLEOD & MOYNE, P.C., 2190 COMMONS PARKWAY, OKEMOS, MI, 48864, US

(21) Appl. No.: 10/705,622

(22) Filed: Nov. 10, 2003

Related U.S. Application Data
(60) Provisional application No. 60/425,450, filed on Nov. 12, 2002.

(51) Int. Cl. A61F 5/44

(52) U.S. Cl. 604/349

(57) ABSTRACT

A device for absorbing liquid due to mild or moderate male incontinence or post urinary drip. The device includes a waist strap, a pouch, a pocket and a insert for the pocket. The waist strap is secured around the waist of the user so that the pouch extends down along the front of the user and the pocket is adjacent the penis of the user. The pocket can contain an insert for absorbing the liquid. The device can be easily moved aside to allow the user to urinate in a standard, upright position.

### CROSS-REFERENCES TO RELATED APPLICATIONS

This application claims the benefit of U.S. Provisional Application No. 60/425,450, filed Nov. 12, 2002.

### STATEMENT REGARDING FEDERALLY SPONSORED RESEARCH OR DEVELOPMENT

Not Applicable

### BACKGROUND OF THE INVENTION

(1) Field of the Invention

The present invention relates to a device for use by men having mild or moderate incontinence. In particular, the invention relates to a device which is secured around a user's waist and which has a pouch with a pocket containing an insert to absorb small amounts of liquid which leaks from a user's penis due to mild or moderate incontinence.

(2) Description of the Related Art

The related art has shown various devices which are secured around the waist of a male user having an incontinence condition to prevent urine from soaking and staining undergarment and clothing. Illustrative are U.S. Pat. Nos. 5,478,334 to Bernstein and 6,223,751 B1 to Park.

Bernstein describes a urine collection assembly which includes a urostomy pouch connected to the waist or hips of a user by a belt. Park describes a incontinence device which includes a cup having a urine absorbing pad. The cup is connected to the user's waist using a waist belt. Both of these devices are bulky and would be visible under a user's clothing. The devices are also intended to collect large amounts of liquid.

The related art has also shown incontinence devices which use a waist strap in combination with other straps to hold the incontinence pocket on the penis. Illustrative are U.S. Pat. Nos. 2,024,341 to DeGraff and 5,275,592 to Grizzaffi which show devices for incontinent males which include straps extending around the waist and the legs of the user to hold the pocket adjacent the penis. Similarly, U.S. Pat. No. 4,387,726 to Denard describes a urine collection device which includes a combined pubic arch support and belt, a combined scrotal support and belt and a urine collecting structure. The above devices prevent movement of the pocket away from the penis of the user. These types of devices do not allow the user to easily perform upright urination without the removal of extra clothing.

Also of interest are U.S. Pat. Nos. 6,129,719 to Nozaki et al. and 6,338,729 to Wada et al. which describe urine absorbent pads which are secured around a user's penis.

Also of some interest is U.S. Pat. No. 6,419,655 B1 to Cohen which describes a male undergarment having a cage removably mounted in the inner side of the front panel of the undergarment. The cage is constructed of a resilient plastic and has a top opening and a hollow interior for receiving a disposable, flexible and watertight pocket. The pocket contains an absorbent material. This device is very bulky and not easily concealed under the user's clothing and would not be comfortable to wear.

Only of minimal interest are U.S. Pat. Nos. 4,700,714 to Fuisz and 5,695,485 to Duperret et al. Fuisz describes a urine collection device which is intended to be secured by adhesive to the inside of a diaper. Duperret et al. describes a pouch which attaches to the underwear of the user by adhesive strips. The pouch includes an outer impermeable membrane, a middle layer of highly absorbent cellulose polyethylene fibers with sodium polyacrylate or similar materials and an inner layer of a permeable, non-absorbent membrane of soft cellulose material. The penis of the male is channeled into the pouch by means of a padded dip in the side of the pouch adjacent the pelvic area of the wearer.

There remains the need for a device for use by men having mild or moderate incontinence which can be concealed under clothing of the user, which is comfortable to wear and which easily allows upright urination by the user.

### SUMMARY OF THE INVENTION

A device for absorbing liquid due to mild or moderate male incontinence or post urinary drip. The device is easily concealed underneath the clothing of the user and is comfortable to wear. The device can be easily adjusted to allow the user to urinate in the upright position without having to remove any articles of clothing.

The device includes a waist strap, a pouch, a pocket and a insert for the pocket. The waist strap is secured around the waist of the user so that the pouch extends down along the front of the user and the pocket is adjacent the penis of the user. The front side of the pocket can have a notch to allow for easy insertion of the penis into the pocket. The notch increases the comfort of the user while wearing the device. The pocket can contain an insert for absorbing the liquid. The insert can be several sheets of tissue which would absorb small amounts of liquid such as in the case of mild incontinence or post urinary drip. The insert could also include moisture and odor absorbing granuals or other materials which absorb larger amounts of liquid. The inserts can absorb up to 8 ounces of liquid. The device is easily movable to reduce the interference caused by the device during upright urination by the user. The pouch and pocket of the device can be easily moved aside to allow the user to urinate in a standard, upright position. The device can be rotated to move the pouch and pocket away from the penis or the pouch can be flipped up out of the way of the penis.

The present invention relates to a device for absorbing liquid due to mild or moderate incontinence, which comprises a waist strap configured to be secured around a waist of a user; a pouch having a first end and a second end with a first surface and a second surface extending therebetween and connected at the first end to the waist strap; a pocket provided at the second end of the pouch adjacent the second surface of the pouch and configured to receive a penis of the user; and an insert for positioning in the pocket for absorbing a small amount of liquid leaking from the penis of the user.

Further, the present invention relates to a method for preventing liquid escaping from a user due to mild or moderate incontinence, from soaking clothing of the user, which provides the steps of providing a device including a waist strap configured to be secured around a waist of the user; a pouch having a first end and a second end with a first surface and a second surface extending therebetween and connected at the first end to the waist strap; a pocket at the second end of the pouch adjacent the second surface of the pouch and configured to receive a penis of the user; and an insert for positioning in the pocket for absorbing a small amount of liquid leaking from the penis, of the user; positioning the device on the user so that the waist strap is about the waist of the user and the pouch extends adjacent the penis of the user; positioning the penis of the user so that at least an end of the penis is located in the pocket of the device; and removing and replacing the insert in the pocket after the insert has absorbed liquid leaked from the penis.

Still further, the present invention relates to a method for preventing liquid escaping from a penis of a user due to mild or moderate incontinence of the user from soaking clothing of the user without interfering with upright urination by the user, which comprises the steps of providing a device having a waist strap configured to be secured around a waist of the user; a pouch having a first end and a second end with a first surface and a second surface extending therebetween and connected at the first end to the waist strap; a pocket at the second end of the pouch adjacent the second surface of the pouch and configured to receive the penis of the user; and an insert for positioning in the pocket for absorbing a small amount of liquid; positioning the device on the user so that the waist strap is about the waist of the user and the pouch extends adjacent the penis of the user; positioning the penis of the user so that at least an end of the penis is located in the pocket of the device; positioning the clothing on the user wherein when liquid leaks from the penis of the user, the liquid is absorbed by the insert and does not escape the pocket of the device and soak the clothing of the user; unfastening the clothing of the user to enable access to the penis of the user; removing the penis of the user from the pocket; and directing the penis toward the waste receptacle.

The substance and advantages of the present invention will become increasingly apparent by reference to the following drawings and the description.

### BRIEF DESCRIPTION OF THE DRAWING(S)

FIG. 1 is a front view of the device 10 showing the insert 20.

FIG. 2 is a back view of the device 10 showing the open top 16C of the pocket 16.

FIG. 3 is a side view of the device 10 showing the user's penis 100 in the pocket 16 and showing the insert 20 at the bottom 16D of the pocket 16.

### DESCRIPTION OF THE PREFERRED EMBODIMENT(S)

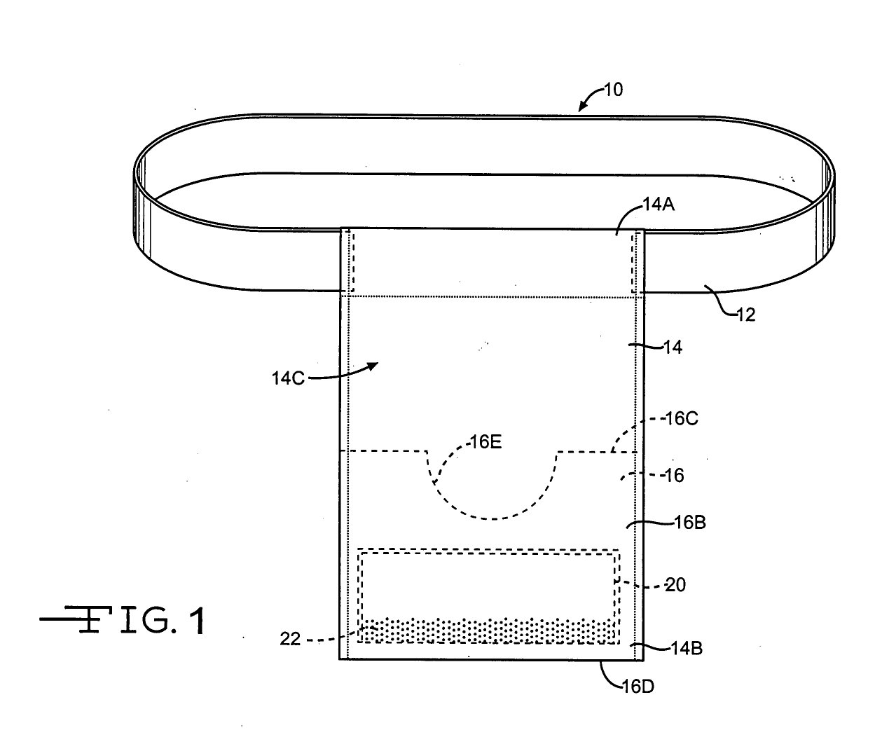

The device 10 of the present invention is intended to be used to conceal the effects of male incontinence by absorbing small amounts of liquid before the liquid reaches the user's clothing. The device 10 of the present invention includes a waist strap, band or belt 12, a pouch 14 and a pocket 16 at one end of the pouch 14 (FIG. 1). An insert can also be provided to be positioned in the pocket 16 to absorb the moisture and control the odor in the pocket 16. The waist strap 12 is configured to be positioned around the waist of the user. In one (1) embodiment, the waist strap 12 is constructed of an elastic material and is formed as a continuous loop. The size of the waist strap 12 depends on the size of the waist of the user. The waist strap 14, in one (1) embodiment, fits snug around the waist of the user without being uncomfortable for the user. In one (1) embodiment, the waist strap 14 is of such a size that the device 10 is easily rotated about the waist of the user without discomfort to the user. In one (1) embodiment, the waist strap is of such a size that the device 10 is only able to be rotated a small distance around the waist of the user so as to move the pouch 14 away from th penis 100 of the user. In another embodiment (not shown), the waist strap 12 is constructed of a non-elastic material and the waist strap 12 has an adjustment mechanism which allows for tightening the waist strap 12 around the waist of the user. In this embodiment, a single size waist strap 12 can be used for a variety of sized users. In one (1) embodiment, the waist strap 12 has opposed ends with each ennd secured to a side of the pouch 14 at the top of the pouch 14.

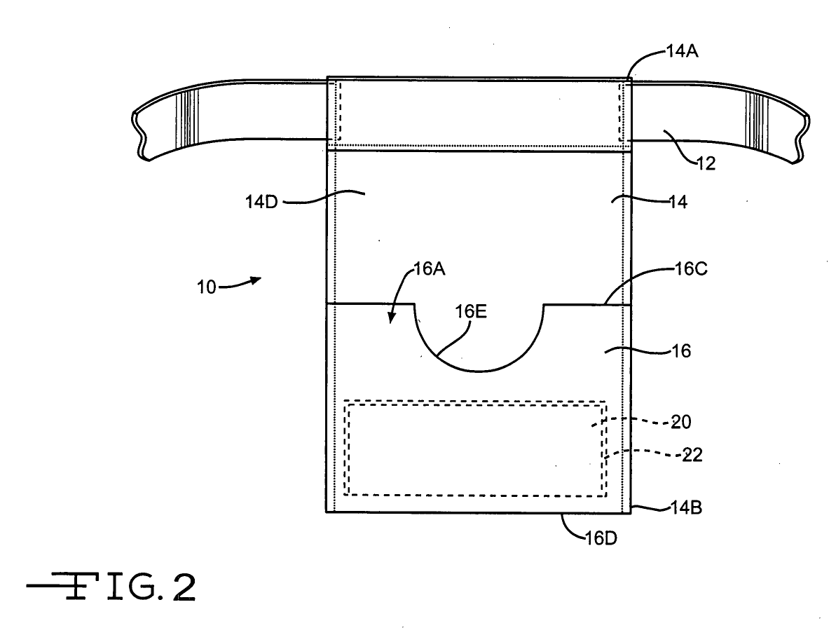

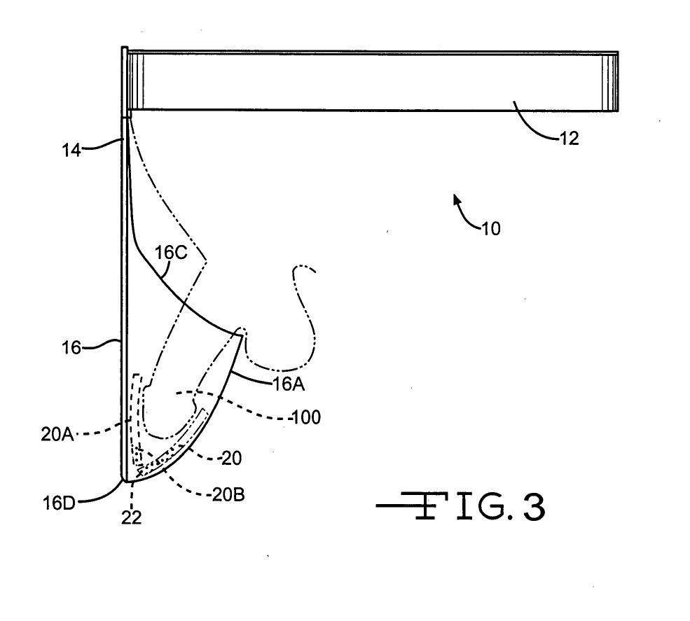

The pouch 14 is secured to the waist strap 12 and extends downward from the strap 12 along the front of the user. The pouch 14 has an essentially rectangular shape with a top end 14A, a bottom end 14B, and having an outer surface 14C and an inner surface 14D extending therebetween. The top or first end 14A of the pouch 14 is connected to the waist strap 12. The top end 14A of the pouch 14 can extend over the waist strap 12 to form a flap between the user and the waist strap 12 to increase the comfort of the user. A pocket 16 is formed adjacent the bottom end 14B of the pouch 14 on the inner surface 14D of the pouch 14. The pocket 16 has a front side 16A and a back side 16B with a closed bottom 16D adjacent the bottom end 14B of the pouch 14 and an open top 16C spaced apart from the bottom end 14B of the pouch 14 toward the top end 14A of the pouch 14. In one (1) embodiment, the pouch 14 and pocket 16 are formed as a unitary piece. In this embodiment, the pocket 16 is formed by folding the bottom end 14B of the pouch 14 inward and upward and securing the sides of the pouch 14 together so that the inner surface 14D of the pouch 14 forms the inner side of the front and back sides 16A and 16B of the pocket 16 and so that a bottom fold forms the closed bottom 16D of the pocket 16. The front side 16A of the pocket 16 is provided with a notch 16E along the top edge (FIG. 2). In one (1) embodiment, the notch 16E has a semi-circular shape. The notch 16E allows for easier positioning of the penis 100 in the pocket 16 and increases the comfort of the user (FIG. 3). The length and width of the pouch 14 depends on the size of the user. However, the length of the pouch 14 is such that the end of the user's penis 100 can be fully positioned in the pocket 16. The pouch 14 and pocket 16 are constructed of a lightweight, breathable material which is non-abrasive to the skin of the user and which keeps moisture away from the skin of the user. The pouch 14 and pocket 16 can be constructed of laminated and coated fabrics. In one (1) embodiment, the waist strap 12, pouch 14 and pocket 16 are reusable and washable.

The device 10 is preferably provided with an insert 20 to absorb liquid escaping into the pocket 16. In one (1) embodiment, the insert 20 has a front sheet 20A and a back sheet 20B with a moisture and odor absorbing material 22 sandwiched therebetween (FIG. 3). The front and back sheets 20A and 20B can be secured together around the perimeter of the sheets 20A and 20B. In one (1) embodiment, the front and back sheets 20A and 20B are formed by a single sheet which is folded. In one (1) embodiment, the insert 20 has a rectangular shape with a longitudinal length between the ends of less than the width of the pocket 16. The insert 20 has an inner chamber for holding the moisture and odor absorbing material 22. The front and back sheets 20A and 20B are constructed of a permeable material which allows moisture and odors to pass through the front or back sheet 20A and 20B into the inner chamber and into contact with the moisture and odor absorbing material 22. In one (1) embodiment, the front and back sheets 20A and 20B are constructed of a light-weight, two-phased heatable tissue comprised of a special blend of thermostatic fiber. In one (1) embodiment, the moisture and odor absorbing material 22 is a lightly cross-linked sodium polyacrylate such as sold under the name ABSORB-Q-GEL and manufactured by Pioneer Medical, Inc. of Lakeland, Fla. In this embodiment, the insert 20 is provided with enough moisture and odor absorbing material 22 to absorb about 8 ounces (1 cup) of liquid. It is understood that less moisture and odor absorbing material 22 may be used in the insert 20. It is understood that other moisture absorbing materials well known in the art could also be used. The moisture and odor absorbing material 22 is provided in a crystalized or granular form such that the material 22 is able to move around the inner chamber of the insert 20. In one (1) embodiment, the insert 20 has a minimum thickness between the front and back sides of the insert 20. In one (1) embodiment, the insert 20 has a thickness of approximately 0.015625 inch (0.039688 cm) without the moisture and odor absorbing material 22 and approximately 0.0625 inch (0.1588 cm) with a standard amount of the moisture and odor absorbing material 22 between the front and back sides 20A and 20B. In one (1) embodiment, the insert 20 is sheets or layers of materials which absorb liquid such as tissue.

To use the device 10, the insert 20 is positioned in the inside of the pocket 16 along the closed bottom 16B of the pocket 16. The insert 20 could be positioned in the pocket 16 after the device 10 is positioned on the user. In one (1) embodiment, the insert 20 is folded along its longitudinal length. The insert 20 is then positioned in the bottom 16D of the pocket 16 such that the fold of the insert 20 is positioned adjacent to and along the fold at the bottom 16D of the pocket 16. Thus, when the insert 20 is correctly positioned in the pocket 16, the insert 20 extends along the closed bottom 16D of the pocket 16 and extends upward from the closed bottom 16D along the front and back sides 16A and 16B of the pocket 16. The insert 20 can be lightly shook before being positioned to distribute the moisture and odor absorbing material 22 along the length of the insert 20. It is understood that more than one (1) insert 20 can be used simultaneously in the pocket 16. Once the insert 20 is in place, the waist strap 12 of the device 10 is then positioned about the waist of the user. The waist strap 12 is securely and snugly fit around the waist of the user to hold the pouch 14 in position. However, the waist strap 12 is comfortable for the user to wear for extended periods of time. When the device 10 is correctly positioned on the user, the pouch 14 extends downward from the waist of the user along the front of the user such that the inner side 14E of the pouch 14 is adjacent the user and the front side 16A of the pocket 16 is adjacent the penis 100 of the user. It is understood that the position of the pouch 14 on the user can be adjusted by rotating the waist strap 12 around the waist of the user. Once the device 10 is correctly positioned, the penis 100 of the user is positioned in the pocket 16 such that the end of the penis 100 is adjacent the insert 20. In the one (1) embodiment having the notch 16E in the front side 16A of the pocket 16, the penis 100 is positioned in the notch 16E. Finally, the user puts on clothing to conceal the device 10. The size, shape and flexibility of the device 10 enables the device 10 to be easily concealed under normal clothing. The materials used to construct the device 10 allow the device 10 to be worn comfortably for long periods of time.

When liquid leaks or drips from the penis of the user due to mild or moderate incontinence or due to post urinary drip, the insert 20 in the pocket 16 acts to absorb the liquid. The device 10 absorbs small amounts of liquid preventing the liquid from soaking the clothing of the user thus preventing embarrassment to the user. In one (1) embodiment, the device 10 does not prevent greater than 4 ounces of liquid from escaping from the device 10 and soaking a user's clothing. In one (1) embodiment the insert 20 of the device 10 absorbs no greater than one (1) ounce of liquid. In one (1) embodiment, the device 10 can prevent up to and including 8 ounces of liquid from escaping the pocket 16 and soaking and staining a user's clothing. After the insert 20 has absorbed the liquid, the insert 20 can be easily removed and replaced. To remove the insert 20, the clothing of the user is unfastened to allow access to the device 10. Next, the penis 100 of the user is removed from the pocket 16 to allow access to the insert 20. Once the insert 20 is removed and replaced, the penis 100 of the user is replaced in the pocket 16 and the clothing of the user is refastened.

The user can easily urinate in the upright position while wearing the device. The structure of the device 10 enables the device 10 to be easily moved and repositioned about the waist of the user so as to reduce the interference of the device 10 to allow upright urination by the user. To urinate, the clothing of the user is unfastened to allow access to the device 10. Next, the penis 100 of the user is moved out of the pocket 16 of the device 10. If necessary, the pouch 14 is moved out of the way by simply lifting the pouch 14 upward. Alternately, the pouch 14 can be moved out of the way by rotating the device 10 about the waist of the user. Once the device 10 is no longer an obstruction, the penis 100 of the user is directed toward the waste receptacle. When the user has finished urinating, the device 10 is repositioned so that the pocket 16 is adjacent the penis 100 of the user. The penis 100 of the user is then inserted into the open top 14A of the pocket 14 and the clothing of the user is refastened.

It is intended that the foregoing description be only illustrative of the present invention and that the present invention be limited only by the hereinafter appended claims.

I claim:

1. A device for absorbing liquid due to mild or moderate incontinence, which comprises:
(a) a waist strap configured to be secured around a waist of a user;
(b) a pouch having a first end and a second end with a first surface and a second surface extending therebetween and connected at the first end to the waist strap;
(c) a pocket provided at the second end of the pouch adjacent the second surface of the pouch and configured to receive a penis of the user; and
(d) an insert for positioning in the pocket for absorbing a small amount of liquid leaking from the penis of the user.

2. The device of claim 1 wherein the waist strap is configured to fit closely around the waist of the user and to be rotatable about the waist of the user.

3. The device of claim 1 wherein the pouch is secured to the waist strap so that the second surface of the pouch is adjacent the user, wherein the pouch and the pocket are a unitary piece and wherein the pocket is formed by folding the second end of the pouch toward the first end of the pouch.

4. The device of claim 1 wherein the first end of the pouch extends around the waist strap so that the pouch is positioned between the waist strap and the waist of the user.

5. The device of claim 1 wherein the pocket includes a first side and a second side with an open first end and a closed second end and wherein the first side of pocket at the first end has a notch.

6. The device of claim 1 wherein the pocket is spaced apart from the waist strap.

7. The device of claim 1 wherein the insert is tissue.

8. The device of claim 1 wherein the insert includes a front side and a back side with a moisture absorbing material spaced therebetween.

9. The device of claim 8 wherein the material is a lightly cross-linked sodium polyacylate.

10. The device of claim 8 wherein the front side and the back side are constructed of light-weight, two-phased heatable tissue.

11. The device of claim 10 wherein the tissue includes a blend of thermostatic fiber.

12. The device of claim 1 wherein the insert has a thickness of about 0.0625 inches (0.1588 cm).

13. The device of claim 1 wherein the insert has a thickness of about 0.015625 inches (0.039688 cm).

14. The device of claim 1 wherein the waist strap, pouch and pocket are constructed of a washable material.

15. The device of claim 1 wherein the pouch and pocket are constructed of a breathable material.

16. The device of claim 1 wherein the pocket is constructed of a waterproof material.

17. The device of claim 1 wherein the insert is able to absorb no greater than 8 ounces of liquid.

18. The device of claim 1 wherein the insert is able to absorb no greater than 4 ounces of liquid.

19. The device of claim 1 wherein the insert is able to absorb no greater than 1 ounce of liquid.

20. A method for preventing liquid escaping from a user due to mild or moderate incontinence, from soaking clothing of the user, which provides the steps of:
(a) providing a device including a waist strap configured to be secured around a waist of the user; a pouch having a first end and a second end with a first surface and a second surface extending therebetween and connected at the first end to the waist strap; a pocket at the second end of the pouch adjacent the second surface of the pouch and configured to receive a penis of the user; and an insert for positioning in the pocket for absorbing a small amount of liquid leaking from the penis of the user;
(b) positioning the device on the user so that the waist strap is about the waist of the user and the pouch extends adjacent the penis of the user;
(c) positioning the penis of the user so that at least an end of the penis is located in the pocket of the device; and
(d) removing and replacing the insert in the pocket after the insert has absorbed liquid leaked from the penis.

21. The method of claim 20, wherein further in step (d), the device is rotated about the waist of L the user so that the pocket is misaligned with the penis of the user and the penis of the user is easily removed from the pocket to allow access to the insert.

22. The method of claim 20 wherein after step (c), clothing put on by the user conceals the device and wherein further in step (d), the clothing of the user is unfastened to allow access the device.

23. The method of claim 20 wherein the pocket has a front side with a notch at a first end and wherein in step (c), the penis is positioned in the pocket through notch.

24. A method for preventing liquid escaping from a penis of a user due to mild or moderate incontinence of the user from soaking clothing of the user without interfering with upright urination by the user, which comprises the steps of:
(a) providing a device having a waist strap configured to be secured around a waist of the user; a pouch having a first end and a second end with a first surface and a second surface extending therebetween and connected at the first end to the waist strap; a pocket at the second end of the pouch adjacent the second surface of the pouch and configured to receive the penis of the user; and an insert for positioning in the pocket for absorbing a small amount of liquid;
(b) positioning the device on the user so that the waist strap is about the waist of the user and the pouch extends adjacent the penis of the user;
(c) positioning the penis of the user so that at least an end of the penis is located in the pocket of the device;
(d) positioning the clothing on the user wherein when liquid leaks from the penis of the user, the liquid is absorbed by the insert and does not escape the pocket of the device and soak the clothing of the user;
(e) unfastening the clothing of the user to enable access to the penis of the user;
(f) removing the penis of the user from the pocket; and
(g) directing the penis toward the waste receptacle.

25. The method of claim 24 wherein after step (f), the pouch is flipped upward away from the penis of the user.

26. The method of claim 24 wherein further in step (f), after the penis is removed from the pocket, the device is rotated about the waist of the user so that the pouch is not adjacent the penis and does not interfere with the urination activities of the user and wherein upon completion of urination, the device is rotated about the waist of the user so that the pouch is adjacent the penis of the user, the penis is positioned in the pocket and the clothing is refastened.
